(19) United States
(12) Patent Application Publication
(10) Pub. No.: US 2016/0271167 A1
(43) Pub. Date: Sep. 22, 2016

(54) METHOD FOR DETECTION TREATMENT AND PREVENTION OF NEUROLOGICAL DEVELOPMENT DISORDERS

(71) Applicant: Shui Yin Lo, Pasadena, CA (US)

(72) Inventor: Shui Yin Lo, Pasadena, CA (US)

(21) Appl. No.: 15/074,178

(22) Filed: Mar. 18, 2016

Related U.S. Application Data
(60) Provisional application No. 62/136,426, filed on Mar. 20, 2015.

(51) Int. Cl. A61K 33/00; A61K 9/00; A61K 9/06; A61B 5/00; A61B 5/16; A61H 39/02; A61M 5/178; A61M 15/00; A61M 16/14; A61K 9/14; A61B 5/01

(52) U.S. Cl. CPC A61K 33/00; A61K 9/14; A61K 9/0078; A61K 9/0019; A61K 9/0046; A61K 9/0048; A61K 9/06; A61B 5/0075; A61B 5/015; A61B 5/4839; A61H 39/02; A61M 5/178; A61M 15/00; A61M 16/14; A61B 5/16; A61B 2503/06

(57) ABSTRACT

A method for detection, treatment, and prevention of neurological development disorders utilizes an infrared imaging device, meridian theory of traditional Chinese medicine, and predetermined amounts of solid water particles to detect, treat, and prevent a neurological developmental disorder, such as autism. The method detects the disorder by initially identifying at least one hot spot on a patient with an infrared imaging device. At least one acupoint along a meridian pathway is identified. The hot spot is correlated with at least one acupoint located along the meridian pathway. The nexus of the hot spot and the meridian pathway indicates a potential neurological development disorder. A degree of irregularity of the internal organ is discerned by measuring a quantitative temperature at the nexus. A solid water particle is applied through a delivery means, such as oral, topical, vapor, and intravenous administration for treatment of the neurological developmental disorder.

### FIELD OF THE INVENTION

The present invention relates generally to a method for detection, treatment, and prevention of neurological development disorders. More so, a method for detection, treatment, and prevention of neurological development disorders utilizes an infrared imaging device for imaging at least one hot spot on a body to indicate internal organ irregularities, a meridian theory of traditional Chinese medicine for identifying at least one acupoint along a meridian pathway, and a delivery means to administer predetermined amounts of solid water particles to a nexus between the hot spot and the acupoint in different regions of the body.

### BACKGROUND OF THE INVENTION

Typically, neurological development disorders are impairments of the growth and development of the brain or central nervous system. Specifically, the neurological development disorders comprises a disorder of brain function that affects emotion, learning ability, self-control and memory and that unfolds as the individual grows. Neurodevelopmental disorders are associated with widely varying degrees of difficulty, which may have significant mental, emotional, physical, and economic consequences for individuals, as well as for their families and society in general.

It is known that normal and abnormal behavior are differentiated by subtle, complex patterns of activity that an expert clinician observes or discovers through systematic diagnostic tests. In practice, the vast majority of pediatric neuropsychiatric and neurological assessment is based on observing behaviors or by asking caregivers about the child in an effort to understand brain function. Such assessment is particularly difficult in infants and young children who may exhibit a limited set of behaviors and limited communication abilities.

It is recognized that, neuroimaging studies link functional brain activity to behavior promises exciting opportunities for measuring nonlinear brain activity that may indicate abnormalities or allow response to therapy to be monitored. Measurements of brain electrical activity with electroencephalography (EEG) have long been a valuable source of information for neuroscience research, yet this rich resource may be under-utilized for clinical applications in neurology and psychiatry. To fully exploit this data, methods for discovering subtle patterns in nonlinear features and deeper understanding of the relationship between emergent signal features and the underlying neurophysiology are needed.

It is recognized that the most recent disorder that has gained much public awareness is Autistic Spectrum Disorder (“ASD”). The number of children that suffer from ASD is increasing at an alarming rate. Unfortunately, there is no known cure for ASD. Some researchers argue that ASD might be related to gastro-intestinal diseases, while others suggest that it may be an autoimmune disease, and still others advocating a viral causation.

It is recognized that pediatricians often treat children with ASD notice that most of the children with ASD have gastro-intestinal problems. However, the definition of a neurological development disorder implies that the symptoms are mainly in the brain. Whatever the actual source may be, treating ASD as solely a problem of the brain may be too limited.

Typically, traditional Chinese medicine has used acupuncture and qi-gong massage to treat ASD with varying degrees of success. Much of traditional Chinese medicine is based on the meridian theory. Meridians are a discrete, non-physical network that connects different parts of the body together, namely internal organs. It creates a comprehensive map in the body that creates a channel for the organs to communicate and channel energy, or qi. Along the meridian path, there are acupuncture points.

It is known that in meridian theory and acupuncture, there are the yin and yang meridians. The yang meridian include regions surrounding various acupoints along the gallbladder (GB), bladder (BL), stomach (ST), small intestine (SI), large intestine, and San Jiao (SJ) meridians. These meridians are the yang meridians that start from the head, and hence have branches reaching into the brain, and extend down to the gastrointestinal tract and urinary bladder systems. The extension of each of these meridians affects the organs it corresponds to and the local areas in which they travel.

Other proposals have involved methods for treating neurological development disorders. The problem with these devices is that they do not merge traditional Chinese medicine with modern medicine.

Thus, an unaddressed need exists in the industry to address the aforementioned deficiencies and inadequacies. Even though the above cited methods for detection, treatment, and prevention of neurological development disorders meets some of the needs of the market, a method for detection, treatment, and prevention of neurological development disorders utilizes an infrared imaging device, meridian theory of traditional Chinese medicine, and predetermined amounts of solid water particles to detect, treat, and prevent a neurological developmental disorder, such as autism is still desired.

### SUMMARY OF THE INVENTION

The present invention is directed to a method for detection, treatment, and prevention of neurological development disorders. The method utilizes an infrared imaging device for imaging at least one hot spot on a body to indicate internal organ irregularities, a meridian theory of traditional Chinese medicine for identifying at least one acupoint along a meridian pathway, and a delivery means to administer predetermined amounts of solid water particles to a nexus between the hot spot and the acupoint in different regions of the body.

The method is configured to detect the disorder by initially identifying at least one hot spot on a body. The hot spot is detected by utilizing an infrared imaging device, such as a thermograph, to capture a thermograph image on a portion of a body. The thermograph image may display at least one hot spot on the body. The hot spot may be indicative of an abnormality in an internal organ within the body.

The method also identifies at least one acupoint that is disposed along a meridian pathway of the body. Those skilled in the art will recognize that the meridian pathway comprises a discrete, non-physical network that connects different internal organs of the body. The meridian pathway may also be defined as a line of energy flow from an internal organ to the surface of the body. Those skilled in the art will recognize that the meridian pathway is determined through traditional Chinese medicinal means.

In one possible embodiment, the acupoint, as used in the method, is a specific point on the surface of the body located on the meridian pathway. The acupoint may also be defined as a point on the body into which an acupuncture needle may be inserted for treatment, as is performed in traditional Chinese medicine.

Once the hot spot and the at least one acupoint along the meridian pathway are determined, a nexus where the hot spot at least partially engages the at least one acupoint is determined. The nexus must lie approximately along the meridian pathway.

The nexus between the hot spot and the acupoint indicates a potentially problematic organ, which may be symptomatic of autism. This is because the meridian pathway serves as an optical fiber that channels the heat of internal organs to the surface of the body. The higher body surface temperature indicated by the hot spot is thus, a reflection of the temperature of the internal organs where the meridian pathways pass through.

Specifically, the hot temperature at the acupoint along the meridian pathway is an indication of the inflammation of the internal organs. Those skilled in the art will recognize that inflamed internal organs emit hot infrared radiation, which are transmitted preferentially along the meridian pathways and peaks at known acupoints that are close to the surface of the body. Thus, the method measures the degree of inflammation of the internal organ by the quantitative temperature at the acupoint. In essence, internal organ abnormalities can be revealed by taking thermographs of the body and corresponding problematic organs with the meridian pathways.

Once the nexus between the hot spot and at least one acupoint has been discerned, the treatment commences. The treatment may include administrating a predetermined amount of solid water particle to the body with a delivery system. In one embodiment, the SWP consists of only pure water molecules and no other chemicals, and generally does not have harmful side effects. The SWP may include, without limitation, stable water clusters, polydihydrogenoxide, and I_{E}.

The delivery system comprises at least one of the following: oral administration, topical administration, vapor administration, and intravenous administration. The solid water particle may be administered on the nexus and body through various means, such that healing of the neurological developmental disorder is attempted. One exemplary administration of the solid water particle includes a pregnant mother drinking the solid water particles or utilizing any number of other possible applications of the solid water particles to her child.

One aspect of the method for detection, treatment, and prevention of a neurological development disorder, comprises:
determining at least one hot spot on a body with an infrared imaging device, the at least one hot spot being at least partially indicative of at least one internal organ of the body having an irregularity;
determining at least one acupoint along at least one meridian pathway, the at least one meridian pathway defined by a network that interconnects the at least one internal organ;
identifying a nexus between the at least one hot spot and the at least one acupoint;
discerning a degree of irregularity of the at least one internal organ by measuring a quantitative temperature at the nexus; and
administering a predetermined quantity of solid water particles approximately to the nexus with a delivery means.

In one aspect of the method, the at least one meridian pathway includes at least one member selected from the group consisting of: a bladder meridian, a stomach meridian, a gallbladder meridian, a large intestine meridian, a small intestine meridian, and a tripe heater meridian.

In another aspect, the at least one acupoint includes at least one member selected from the group consisting of: left and right front center ears; left and right inner point of the eyes; left and right neck near the shoulder; meridian lines; left and right forehead temple area;

left and right collar bone area; left and right arm pits, and an SI line segment at the bottom of the neck.

In another aspect, the infrared imaging device comprises a thermograph.

In another aspect, the delivery means includes at least one member selected from the group consisting of: oral administration, topical administration, vapor administration, and intravenous administration.

In another aspect, the oral administration comprises a liquid, a pill, or a gel consisting of the predetermined quantity of solid water particles.

In another aspect, the topical administration comprises a cream consisting of the predetermined quantity of solid water particles.

In another aspect, the vapor administration comprises a nebulizer and an inhaler consisting of the predetermined quantity of solid water particles.

In another aspect, the intravenous administration comprises an intravenous injection consisting of the predetermined quantity of solid water particles.

In another aspect, the cream is applied at least topically to at least one member selected from the group consisting of: the left and right SJ21 front center ears, left and right BL1 inner points of the eyes, left and right GB14 forehead temple, left and right ST12 collar bone, and left and right GB22 arm pits.

In another aspect, the body comprises a body of a child.

In another aspect, the neurological development disorder is an autistic spectrum disorder.

In another aspect, the step of administering a predetermined quantity of solid water particles approximately to the nexus with a delivery means, further includes at least one of the following: administering the liquid, the pill, or the gel to a mother of a baby, who is breast fed by the mother, administering the cream, the liquid, the pill, or the gel directly to the baby.

In another aspect, the method further comprises at least one supplementary treatment, the at least one supplementary treatment including at least one member selected from the group consisting of: acupuncture, herbs, behavioral therapy, speech therapy, drugs, and at least one alternative method prescribed by a medical professional.

One objective of the present invention is to provide Chinese medicinal theories of meridian for detection, treatment, and prevention of neurological development disorders.

Another objective is to utilize a thermograph to identify irregularities in the internal organs.

Another objective is to administer solid water particles towards a specific region of the body.

Another objective is to administer solid water particles by a variety of routes of administration including: oral, intravenous, intramuscular, subcutaneous, intrathecal, topical, inhalation.

Yet another objective is to provide multiple delivery means for administering the solid water particles.

Furthermore, when no symptom of neurological developmental disorder is detected, the sole purpose of administrating of solid water particles is to prevent the child to develop neurological developmental disorder. This especially applies to child under 2 years old.

When a neurological development disorder is suspected, or detected by other means, the sole object is to treat by administration of Solid Particle Particles.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a flowchart diagram of an exemplary method for detection, treatment, and prevention of neurological development disorders, in accordance with an embodiment of the present invention;

FIG. 2 illustrates an exemplary Stomach Meridian and Large Intestine Meridian of three subjects before solid water particle treatment and 15 minutes after solid water particle treatment, in accordance with an embodiment of the present invention;

FIG. 3 illustrates an exemplary Gallbladder meridian and San Jiao meridian within two subjects before solid water particle treatment and 15 minutes after solid water particle treatment, in accordance with an embodiment of the present invention;

FIG. 4 illustrates an exemplary Small Intestine Meridian of a subject before solid water particle treatment and 15 minutes after solid water particle treatment, in accordance with an embodiment of the present invention;

FIG. 5 illustrates an exemplary Bladder Meridian within two subjects before solid water particle treatment and 15 minutes after solid water particle treatment, in accordance with an embodiment of the present invention;

FIG. 6 illustrates an exemplary Armpit-immune system of two subjects before solid water particle treatment and 15 minutes after solid water particle treatment, in accordance with an embodiment of the present invention;

FIG. 7 illustrates an exemplary images of the torso and lower abdominal of three subjects before solid water particle treatment and 15 minutes after solid water particle treatment, in accordance with an embodiment of the present invention; and

FIG. 8 illustrates an exemplary the back, or DU meridian of a subject before (d) solid water particle treatment and 15 minutes after (e) solid water particle treatment, in accordance with an embodiment of the present invention.

Like reference numerals refer to like parts throughout the various views of the drawings.

### DETAILED DESCRIPTION OF THE INVENTION

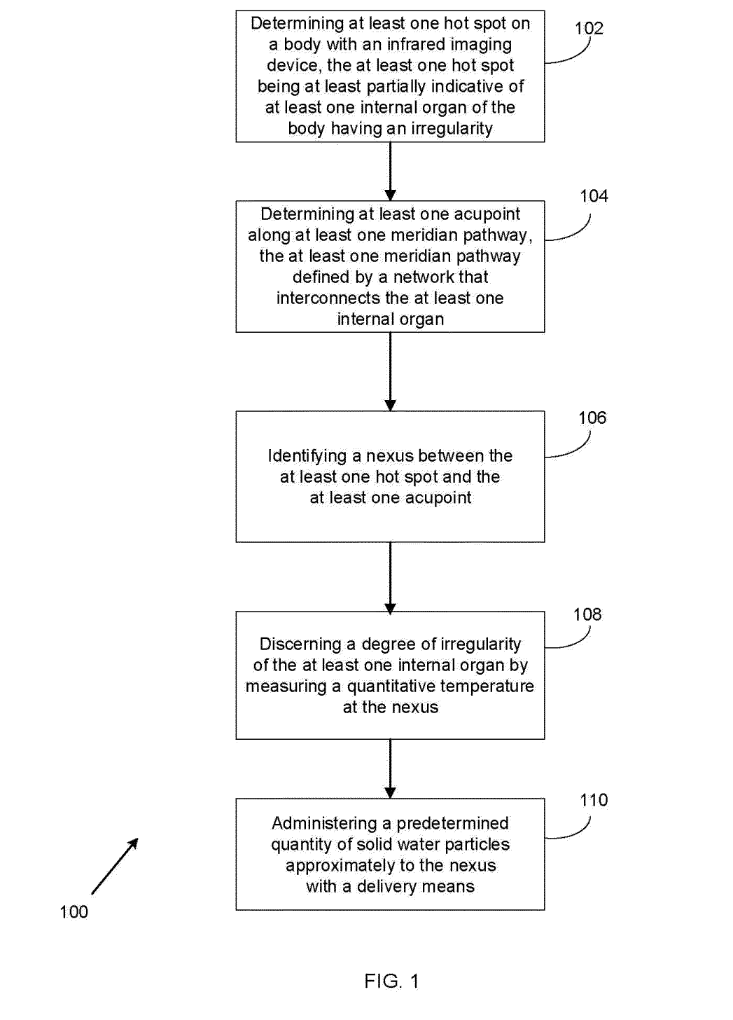

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “first,” “second,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

At the outset, it should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. §112.

In one embodiment of the present invention presented in FIGS. 1-8, a method 100 for detection, treatment, and prevention of neurological development disorders utilizes an infrared imaging device, a meridian theory of traditional Chinese medicine, and a predetermined amount of solid water particles to help detect, treat, and prevent a neurological developmental disorder, such as autism. In one embodiment, the method 100 particularly focuses on the effectiveness of the solid water particles (SWP) on neurological development disorders, such as children with autistic spectrum disorder.

As referenced in the flowchart of FIG. 1, the method 100 may include an initial Step 102 of determining at least one hot spot on a body with an infrared imaging device, the at least one hot spot being at least partially indicative of at least one internal organ of the body having an irregularity. The infrared imaging device may include a thermograph. Those skilled in the art will recognize that the thermograph is an instrument that produces a trace or image representing a record of the varying temperature or infrared radiation over an area or during a period of time. Thermographs, or infrared imaging system, have been in use for many years, mainly in connection with breast cancer detection and imaging.

The method 100 detects the disorder by initially identifying at least one hot spot on a body. The hot spot is detected by utilizing an infrared imaging device, such as a thermograph, to capture a thermograph image on a portion of a body. The thermograph image may display at least one hot spot on the body. The hot spot may be indicative of an abnormality in an internal organ within the body.

The method 100 further includes a Step 104 of determining at least one acupoint along at least one meridian pathway, the at least one meridian pathway defined by a network that interconnects the at least one internal organ. In some embodiments, the at least one meridian pathway may include, without limitation, a bladder meridian, a stomach meridian, a gallbladder meridian, a large intestine meridian, a small intestine meridian, and a tripe heater meridian.

The method 100 also identifies at least on acupoint that is disposed along a meridian pathway of the body. In some embodiments, the at least one acupoint may include, without limitation, a left GB14, a left SJ21, a left BL1, a left ST12, a left GB22, a right GB14, a right SJ21, a right BL1, a right ST12, a right GB22, and an SI line segment at the bottom of the neck.

There are twelve regions that we look for in thermographs to see whether there are hot areas or hot spots, which correspond to acupoints and meridians. The twelve areas, as discussed in Table 1 below, include: left and right front center ears: SJ 21; left and right inner point of the eyes: BL 1; left and right neck near the shoulder: SI meridian lines; left and right forehead temple area: GB 14; left and right collar bone area: ST 12; and left and right arm pits: GB22. In summary these acupoints are defined as six pairs; left and right (SJ21, BL1, SI, GB14, ST12, GB22). There are in addition two hot areas which are related to organs beneath the body surface. They are the gut area, and the reproductive organs.

Those skilled in the art will recognize that the meridian pathway comprises a discrete, network that connects different internal organs of the body. The meridian pathway may also be defined as a line of energy flow from an internal organ to the surface of the body. Those skilled in the art will recognize that the meridian pathway is determined through traditional Chinese medicinal means.

In one possible embodiment, the acupoint, as used in the method, is a specific point on the surface of the body located on the meridian pathway. The acupoint may also be defined as a point on the body into which an acupuncture needle may be inserted for treatment, as is performed in traditional Chinese medicine.

In some embodiments, a Step 106 may include identifying a nexus between the at least one hot spot and the at least one acupoint Once the hot spot and the at least one acupoint along the meridian pathway are determined, a nexus where the hot spot at least partially engages the at least one acupoint is determined. The nexus must lie approximately along the meridian pathway.

It is significant to note that the method 100 lies on the foundation that, based on the meridian theory of traditional Chinese medicine, the body surface temperature phenomena is connected to internal organs. The higher body surface temperature indicated by the hot spot is thus, a reflection of the temperature of the internal organs where the meridian pathways pass through. As measured by the infrared imaging device, hot spots and hot lines on the body surface reflect inflammation of the internal organs,

Thus, the nexus between the hot spot and the acupoint indicates a potentially problematic organ, which may be symptomatic of autism. This is because the meridian pathway serves as an optical fiber that channels the heat of internal organs to the surface of the body. Further, the infrared imaging device provides a non-invasive, passive, thermal detector that can be taken in seconds without disturbing the body's state of health. The thermal image can be taken before and a few minutes after treatment to determine the effectiveness of the treatment. If the treatment for a particular organ is not effective, the temperature along its corresponding meridian will not be changed. Hence, the effectiveness of treatment can be assessed with the infrared imaging device.

A Step 108 comprises discerning a degree of irregularity of the at least one internal organ by measuring a quantitative temperature at the nexus. Specifically, the hot temperature at the acupoint along the meridian pathway is an indication of the inflammation of the internal organs.

Those skilled in the art will recognize that inflamed internal organs emit hot infrared radiation, which are transmitted preferentially along the meridian pathways and peaks at known acupoints that are close to the surface of the body. Thus, the method measures the degree of inflammation of the internal organ by the quantitative temperature at the acupoint. In essence, internal organ abnormalities can be revealed by taking thermographs of the body and corresponding problematic organs with the meridian pathways.

A final Step 110 comprises administering a predetermined quantity of solid water particles approximately to the nexus with a delivery means. Once the nexus between the hot spot and at least one acupoint has been discerned, the treatment commences. The treatment may include administrating a predetermined amount of solid water particle to the body with a delivery system. In one embodiment, the SWP consists of only pure water molecules and no other chemicals, and generally does not have harmful side effects. The SWP may include, without limitation, stable water clusters, polydihydrogenoxide, and I_{E}. The delivery system comprises at least one of the following: oral administration, topical administration, vapor administration, and intravenous administration.

The solid water particle may be administered on the nexus and body through a delivery means, such that healing of the neurological developmental disorder is attempted. The delivery means may include at least one member selected from the group consisting of: oral administration, topical administration, vapor administration, and intravenous administration.

In some embodiments, the oral administration may include a liquid, a pill, or a gel consisting of the predetermined quantity of solid water particles. The topical administration may include a cream consisting of the predetermined quantity of solid water particles. The vapor administration may include a nebulizer and an inhaler consisting of the predetermined quantity of solid water particles. The intravenous administration may include an intravenous injection consisting of the predetermined quantity of SWP.

In one possible embodiment, the mechanism of healing by SWP may be explained simply as follows: meridians are hypothesized to consist of DHW (stable water clusters of double helix shape). The malfunction of organs along meridians is explained in Chinese medicine as the blocking of the flow of qi along the meridians. By drinking SWP, the charged water repairs the meridian and unblocks it, so that qi can freely flow again.

In one embodiment, the delivery system D_{α} that delivers the solid water particles, to the subject under treatment comprises four kinds of delivery systems, which are denoted as D_{α} with α=d, c, b, and v, where “d” means digestive system, “c” means cream, “b” means breathing, and “v” refers to intravenous injection.

The D_{d} intake of SWP to digestive system, which can take the form of drinking a glass of SWP, or pills or gels with SWP. The SWP is taken via the mouth, and primary to the digestive system. Some will be absorbed in the mouth to the blood stream directly.

Further, the D_{c} intake of SWP is carried through the skin via a topical cream, which consists of SWP.

The D_{b} intake of SWP through the breathing, which may be done by nebulizer or inhaler, or any other device. The SWP will go directly upward to the brain and downward to the lung without going through stomach as in the method of D_{d}.

The Dᵥ, intake of SWP through the intravenous method goes directly into the blood stream.

In one exemplary administration, the cream is applied at least topically to at least one member selected from the group consisting of: the left and right front center ears, the left and right inner point of the eyes, the left and right neck near the shoulder, the left and right forehead temple area, the left and right collar bone area, the left and right arm pits, and an SI line segment at the bottom of the neck. In another exemplary administration of the solid water particle a pregnant mother drinks the solid water particles or utilizes any number of other possible administration means of the solid water particles to her child.

The method 100 may include additional steps of administering a predetermined quantity of solid water particles with delivery means, approximately to the nexus. These steps may include administering the liquid, the pill, or the gel to a mother of a baby, who is breast fed by the mother; and administering the cream, the liquid, the pill, or the gel directly to the baby.

Specifically, there are two means in which the prevention of autistic behavior development of a child may be initiated. A first means is, as soon as the baby is born, for the mother to start drinking SWP. In this manner, some of the SWP will be passed to the baby through breast feeding. If the baby is fed with artificial milk, then SWP concentrate can be added to the artificial milk, which may be directly fed to the baby. Another alternative delivery means is the cream with the predetermined amount of SWP that can be applied directly to the skin of the baby with the supervision of a pediatrician. This can be universal to all babies without exception if the family is willing to let the baby be treated with SWP.

The second means is when the baby is ready. In this embodiment, the family must give permission for the infrared thermal scans. Thus, when the thermographs of the baby contain hot spots and hot areas that resemble the symptoms of a child with ASD, treatment with SWP can start immediately with any of the above four methods of delivery systems. It is also significant to note that if the baby does not want to drink any SWP water, the easiest application is the SWP cream.

In alternative embodiment of the method 100, the percentage and the rate of recovery may be improved by adding other therapies such as behavioral therapy, physiological therapy, acupunctures, herbs, drugs, and/or biofeedback, messages, external qi applications, and at least one alternative method prescribed by a medical professional, and other alternative and complementary methods that are suitable to each individual patient.

Provided below are case histories of patients being treated according to the invention which provide evidence of the effectiveness of the treatment methods described herein. The following Examples are intended to illustrate practice of the preferred embodiments of the invention. Numerous additional embodiments and improvements are apparent upon consideration of the following examples.

In an exemplary use of the method 100, parents and guardians with children diagnosed with ASD were recruited by referral as well as by public announcements. This study was conducted in Panama at a medical clinic under the supervision of a licensed medical doctor. There were a total of thirteen autistic children who completed the long term study. The duration of the trial was from one to three months. The average duration of treatment was two months. During the trial period, there were no dietary requirements or changes for each of the subjects.

During the time between the initial visit and the follow up visit, each subject was asked to drink an 8 oz glass of special water mixture twice a day, night, and apply SWP cream twice a day until their follow-up visit. The special water mixture is made by mixing 50 drops of SWP into 1 gallon of distilled water. The mixture was then stirred and shaken to ensure proper mixing. The SWP cream contained 7% SWP.

The subject may be given much more concentrate form of SWP, say 100 to 300 times more, the speed of improvement will be hastened. The improvement is theoretically only the logarithmic dependence of the amount of SWP taken. If one takes 100 times more, the time of improvement will be sped up only several times.

At the first visit each subject's parent was asked to fill out a 10-point health questionnaire, which supplied the quantitative portion of our report. Next, thermographs of the entire body were taken. After the thermographs, each child was given 8 oz of SWP water and had 7% SWP infused cream applied on six standard areas: left and right GB 14 on the forehead, left and right SJ21 on the tragus, and left and right ST12 above the collar bones. After the SWP treatment of water and cream, they were asked to wait 15 minutes. After 15 minutes duration, a second set of thermographs were taken of the entire body. Analysis of the side-by-side comparison of the images before SWP treatment and after the SWP treatment was done.

It is significant to note that in the studies, the change in maximum temperatures from before and after SWP treatment of the acupuncture points where the meridians lie as an indication of the effectiveness of SWP. This criteria is applied to this study in Panama with thirteen ASD subjects. Although the entire body of each subject was taken, we focus on these areas the distribution of surface temperatures on the face, chest, abdomen, front and back of the legs and feet, upper and lower back, the back neck area, and the left and right sides of the neck.

For better understanding and analysis, all thermographs are shown in color with heat differentiation. The temperature range corresponds with the color the thermographs show. The hottest is white, then as the temperature progressively cools; the color pattern goes from red, yellow, green, blue and black as the coldest. The color scale is adjusted so that the green color implied a healthy state and the other colors of yellow, red, and white representing inflamed tissue.

In analyzing all of the thermographs, we focus our attention mainly on the very hot areas, which in our present scheme is red and/or white, with white being the most inflamed. A hot temperature along a meridian line is interpreted to be inflammation of organs and tissues along that meridian, and is considered to be in an unhealthy state.

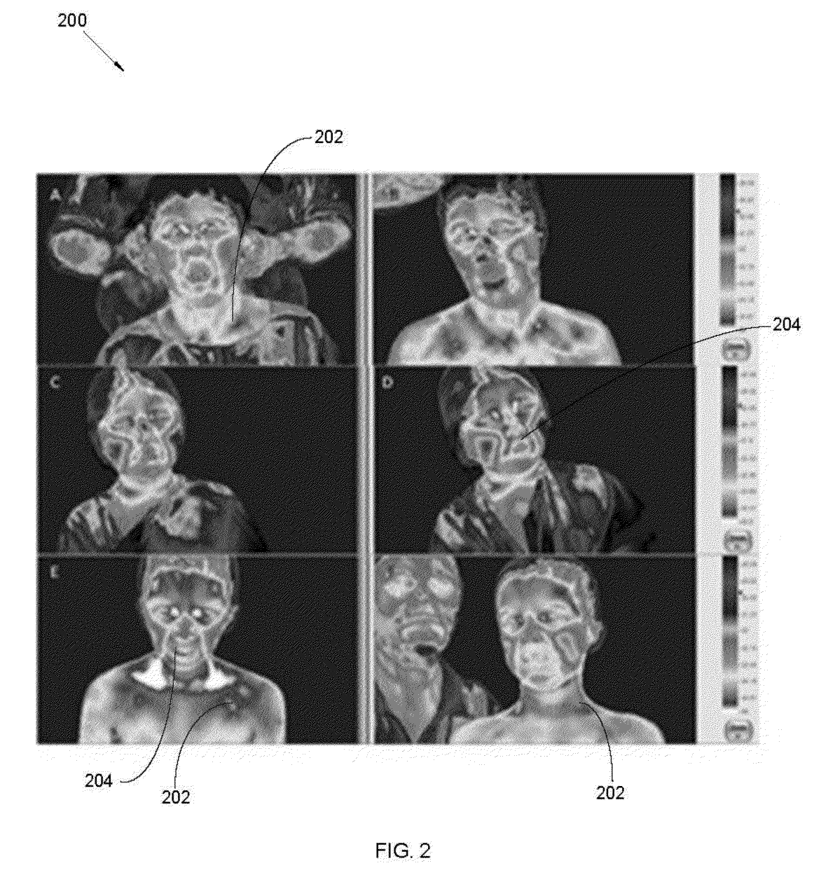

Similarly, in this study the images taken before and after SWP treatment had the same temperature scale to show an accurate analysis of the temperature changes. Thus, images in FIGS. 2-8 show the effects of SWP before and after the treatment. Images taken before the SWP treatment are on the left column. Images taken 15 minutes after SWP treatment are on the right column, which corresponds to its respective “before” images.

In analyzing the thermal images, there are six colors in these thermal images: white, then hottest, then red, yellow, green, blue and black that span 8° C. From one color to another color there is a change of 1.6° C. Within each color there are three distinguishable shades. So, when a different shade of color is observed, it is a change of about 0.5° C. The statistical fluctuation of skin temperature is about 0.1° C. So when a color shade is observed it is five standard deviations away, and, hence, statistically significant, provided that all other conditions remain constant in the 15 minutes duration.

The short term results of the method 100 are as follows: The findings presented some common hot areas seen amongst all the subjects with ASD that we have studied thus far. In this research, we only present the study conducted in Panama on 13 subjects diagnosed with varying degrees of ASD. Some representative examples of the typically seen hot areas of ASD along the six meridians may include: Stomach meridian (ST), Bladder meridian (BL), Gallbladder meridian (GB), Large Intestine meridian (LI), Small Intestine meridian (SI) and San Jiao meridian (SJ). These six meridians are called yang meridians, which are the meridians that connect the head to the digestive and urinary bladder systems.

It is also significant to note that for all diseases associated with the brain, one should look at the conditions of these six meridians. Any cure of brain-related issues should have an effect on some of these six meridians. Alternatively, any healing effect on these six meridians may have an influence on the brain.

FIG. 2 references an example of a thermal image 200 showing a hot spot 202 and acupoint located at the stomach meridians (ST) and a hot spot 204 at the large intestine meridian (LI). FIG. 2 shows three sets of thermography of faces from three ASD subjects. The images on the left (images A, C, and E) were taken before any SWP treatment and the images on the right (B, D, and F) were taken 15 minutes after SWP treatment.

Here, image A shows two red lines running down from below the eyes around the noses down to the opposite sides of the mouth and joined together below the mouth above the chin. They are identified as the ST meridians. Similar red lines can be seen on D and E. It is clear that on the first row, A and B, that there was a cooling effect from the SWP treatment. Thus, the two red lines on image A cools down to a green and yellow color when comparing images A and B. In the second row, C and D show two red faint ST lines. In comparing before image C with after image D the temperature increased from a faint red to a deep red. In the third row, E and F, the two red ST lines cooled down to yellow.

Based on color and temperature analysis as discussed in the Materials and Method section, these three sets of pictures indicate the effect of SWP on ST meridians. Furthermore, there are two red horizontal red lines below the nose and above the mouth on images D and E. These red lines can be easily identified to be the Large Intestine Meridian (LI). For the first subject shown in images A and B, there we do not see the LI meridian, which goes across the upper lip. The LI of the second subject on the second row heats up, and the LI meridian of the third subject cool down.

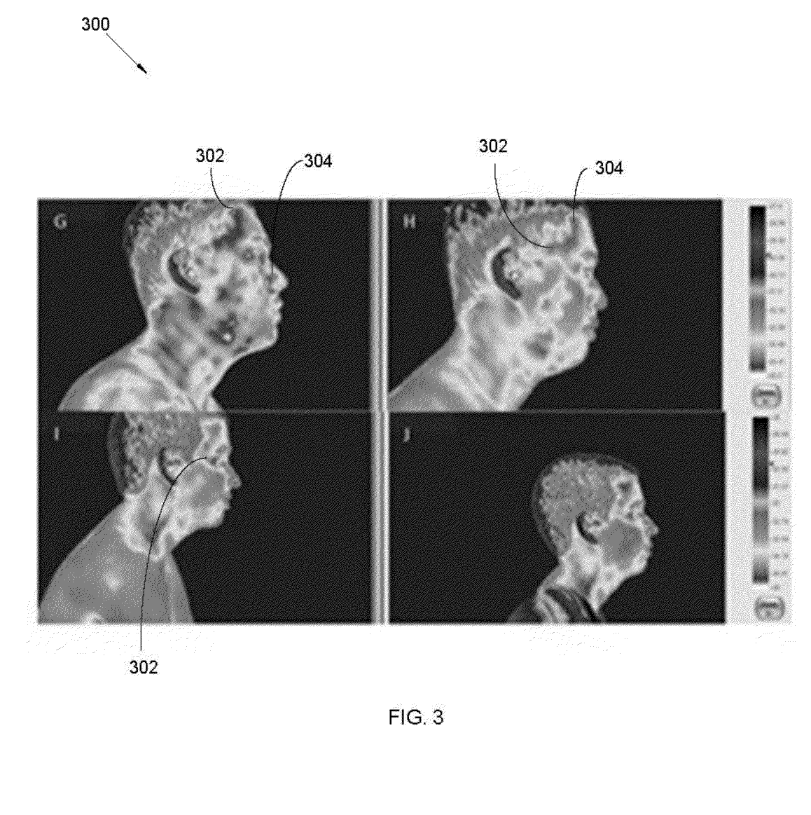

FIG. 3 references an example of a thermal image 300 showing a hot spot 304 and acupoint located at the gallbladder (GB) meridian. FIG. 3 shows a hot spot 302 on the right profile of the head of two subjects. Both subjects presented red areas about an inch above the temples, which can be identified with acupoints GB 14. Next to the two right eyes there are two red spots which is GB1, commonly known as the temple.

Thus, when comparing before and after images G and H, GB14 cooled down from deep red to a lighter red 15 minutes after applying the SWP and drinking SWP water. On the bottom row, images I and J, GB14 turned deep red after applying SWP, opposite of G and H. The white spot on the temple, acupoint GB1, on the top row cools down to red spot, whereas the subject shown on the bottom row the GB 1 hot red spot near the right eye does not change.

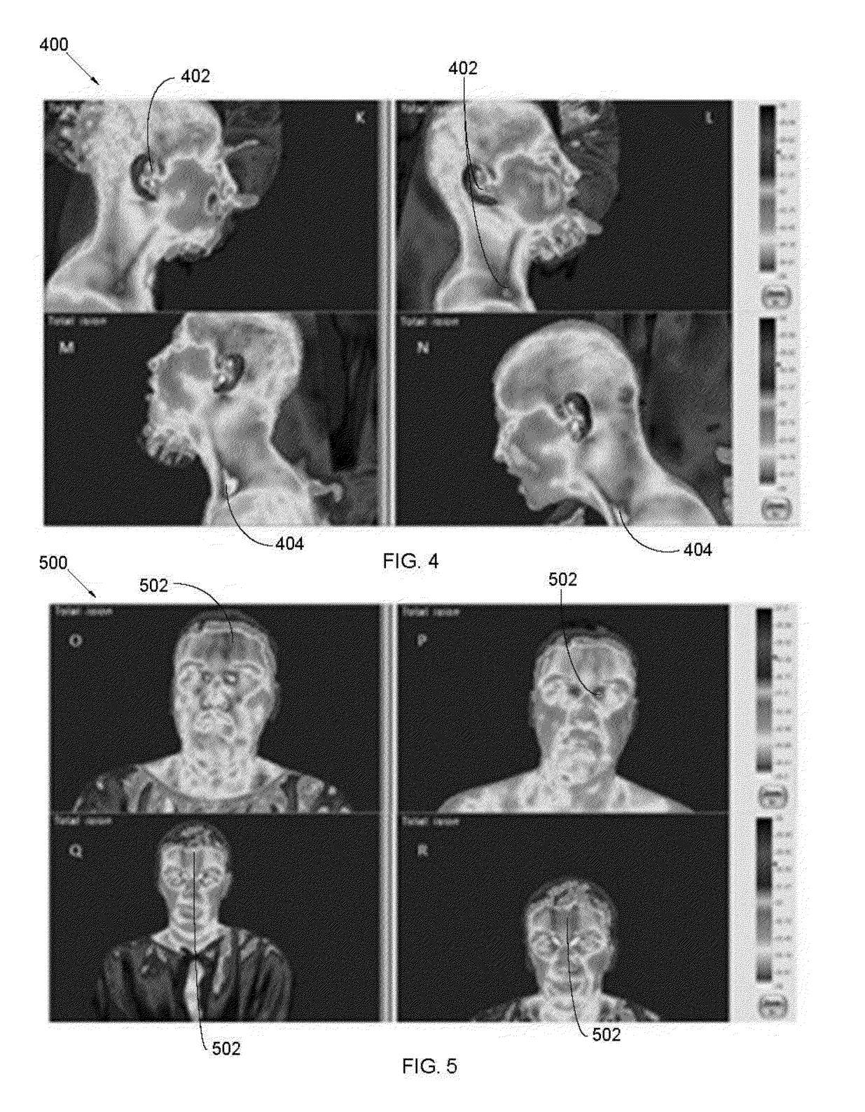

FIG. 4 references an example of a thermal image 400 showing a hot white spot 402 at the acupoints (SJ21) and a hot spot 404 and acupoint located at the small Intestine meridian (SI) and. FIG. 4 shows the thermographs are of the left and right sides of head of a subject. All four pictures presented red vertical lines on the side neck. According to meridian theory and acupuncture they are identified as the Small Intestine meridian (SI). There is no observable color change from image K (before) to image L (after).

However, the white areas in images M and N show that the white area at the end of red SI line shrinks by more than half before (M) and after (N) applying SWP. There are observable white hot spots at the tragus, which are identified as acupoints SJ21. In comparing K and L, the area of the white spot decreases significantly by half. However, we see no noticeable change from M to N.

FIG. 5 references an example of a thermal image 500 showing a hot spot 502 and acupoint located at the bladder meridians (BL). FIG. 5 shows two vertical red lines above the two eyes of both subjects displayed on top and bottom rows are identified as Bladder meridian (BL). The two deep-red BL lines cooled down to light red lines on the top row (0 and P), and the two vertical BL lines heated up to deep-red lines after using SWP on the bottom row (Q and R). The white dots at the inner side of the eyes are acupoints BL1. The inflammations of BL meridians and acupoints BL1 point to unhealthy condition on organs somewhere along the BL meridian line. It is interesting to point out that there are other red lines below the nose and above the mouth which were identified as Large Intestine meridian (LI). In all four images the two red lines down the eyes, along the nose and below the mouth are identified as the Stomach meridian (ST).

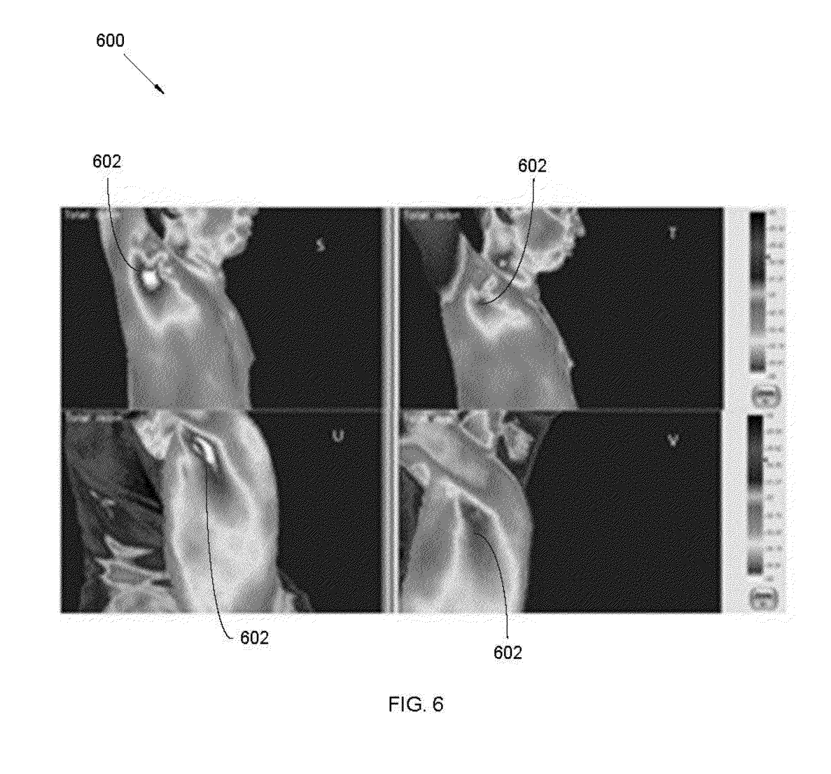

FIG. 6 references an example of a thermal image 600 showing a hot spot 602 and acupoint located at the armpit area. FIG. 6 shows the thermographs of the arm pits area of two subjects. From our study of adult subjects of other health problems we have found a correlation of white area in the armpits related to inflammations of the lymphatic node, or an over activate immune system—with related health issues. Similarly, children with ASD are often found with over active immune system. Hence, some researchers claim that ASD may be caused by malfunction of the immune system. In these images, we see that the hot white areas of both subjects cooled down to become red after using SWP. This shows that SWP has a significant effect on the function of immune system. The reduction of inflammation is regarded as positive influence of SWP on the health of the immune system.

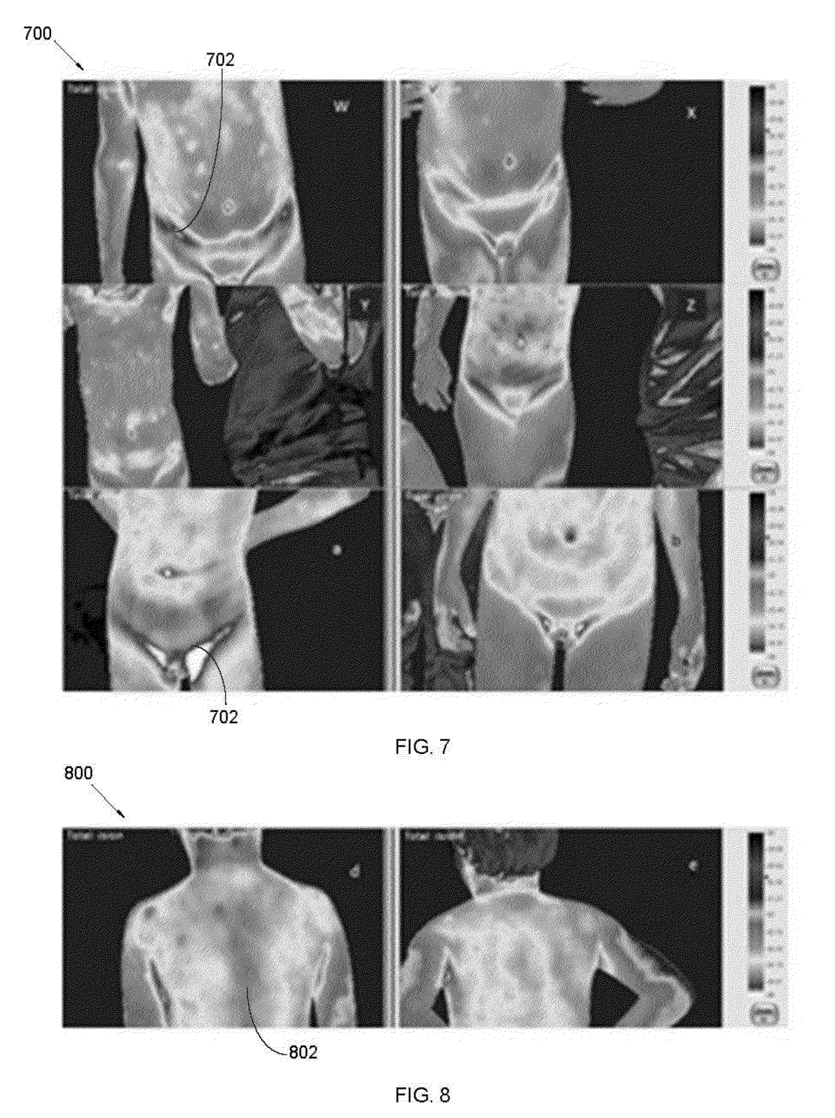

FIG. 7 references an example of a thermal image 700 showing the hot spot 702 and acupoint located at the lower abdominal area. FIG. 7 shows the lower abdominal areas of three subjects. In the top row (W and X) the abdominal area of the subject heated up. The yellow horizontal line in the area above the colon increased in temperature from red to a deeper red after using SWP (Y compared to Z). In the bottom row (a and b) the lower abdominal area of the subject cooled down. The broad horizontal red area corresponds to the colons. The white area around the genitals reduced more than 90% after using SWP.

In a detailed adult study it is apparent that in the first or second minute after using SWP, the hot spots and lines tended to heat up. If the health situation is not serious, it will cool down. However, if the health situation is more serious, then it will become hotter. The adult situation can be applied to those presented ASD children. Our tentative conclusion is that whether the body heats up or cools, it is a positive sign that SWP is working at least in the 15 minutes after using SWP.

FIG. 8 references an example of a thermal image 800 showing the hot spot and acupoint located at the back area. FIG. 8 shows one subject which has a hot red spot 802 that is vertical along the middle of the back. Note that many children have such hot areas at the back. It is clear after drinking SWP that the red area reduces significantly and the green area increases significantly. Significant change is seen throughout the back along the spine.

For long term quantitative results of the method 100, a 10-point health evaluation form may be completed by the parents and guardians of the autistic children to determine the quantitative improvement of their children. The parents fill out the questionnaire during the initial visit and the same questionnaire form is filled out 6-9 weeks after the start of the SWP treatment. They were asked to evaluate their children using a scale from 1 to 10 of unhealthy/non-functional or healthy/functional, respectively.

Based on observations, the parents were asked to assign a number scaled from 1 to 10 each week based the child's level of problems or competence during that week. On the form, a 1 represented the best health condition, and 10 represented the worst. By the end of the long term study the parents were asked to fill the form again. By comparing the final forms with the initial form filled in at the beginning of the trail, a quantitative measurement of the improvement of the child were obtained. The quantitative data is shown in Table 1 above.

The ten areas of evaluation asked in the questionnaire are as follows:
1. Digestive system: eating, appetite, drinking, stomach etc.
2. Bowel movement, urine
3. Body movement: repetitive, obsessive, compulsive
4. Sleeping and waking
5. Communication of needs, following directions
6. Language skills
7. Non-verbal expressions: eye contact, facial expressions, ticks, posture
8. Social skills: interacting with others, peer relationships
9. Attention span or focus
10. Ability to attend to academic studies

Here, sⱼ is the score that a parent gives to the child on question j, where question j runs from 1 to 10 as described above. The range of values for sⱼ is from 1 to 10, 1 being the best condition and 10 being the worst condition. For example s₉=9 meant the child's attention span was very bad. On the other hand s₂=2 meant his bowel movements were very good. Of most interesting would be the improvement that the child achieved during the trial period. So, the important quantity would be the improvement, Δs:

Δsⱼ=sⱼ(1)−sⱼ(12) equation (1)

where sⱼ(1) represented the score at week 1, the beginning of the clinical test, and sⱼ(12), the score at week 12, the end of the clinical test. Questions 1 through 4 were designed to evaluate the physiological state of the child. We denote the average value by

Δₚ=1/4(Δs₁+Δs₂+Δs₃+Δs₄) equation (2)

When Δₚ is positive, it meant the physiological state of the child has improved in the trial period. If Δₚ was negative, it meant the physiological state of the child had deteriorated. Questions 5 through 9 are an evaluation of the child's behavior and developmental state. We denote the average behavior and developmental improvement to be

Δ_{d}=1/6(Δs₅+Δs₆+Δs₇+Δs₈+Δs₉+Δs₁₀) equation (3)

Similarly, when Δ_{d} was positive, it meant that the behavioral and developmental state of the child has improved. Conversely, when Δ_{d} was negative, it meant the behavioral and development state of the child had deteriorated. The results are shown in Table 2 as percentage of improvement of each subject's physiological and behavioral state.

Table 2 is as follows:

| CODE | Initial | Follow-up | Difference | % difference |
|---|---|---|---|---|
| S1 | 29 | 21 | −8 | 72.42 |
| S2 | 41 | 32 | −9 | 78.05 |
| S3 | 15 | 13 | −2 | 86.67 |
| S4 | 65 | 60 | −5 | 92.3 |
| S5 | 51 | 50 | −1 | 98 |
| S6 | 16 | 14 | −2 | 87.5 |
| S7 | 63 | 59 | −4 | 93.65 |
| S8 | 58 | 47 | −11 | 81.03 |
| S9 | 16 | 10 | −6 | 62.5 |
| S10 | 52 | 42 | −10 | 80.77 |
| S11 | 58 | 38 | −20 | 65.52 |
| S12 | 56 | 28 | −28 | 50 |
| S13 | 48 | 25 | −23 | 52.08 |

The mechanism of healing by SWP may be explained simply as follows: meridians are hypothesized to consist of DHW (stable water clusters of double helix shape). The malfunction of organs along meridians is explained in Chinese medicine as the blocking of the flow of qi along the meridians. By drinking SWP, the charged water repairs the meridian and unblocks it, so that qi can freely flow again.

In essence, a blocked meridian is like a river that is blocked by a boulder; the qi cannot flow smoothly. An alternate small river is built around the blockage by DHW. Alternatively, DHW unblocks the boulder and hence qi can flow smoothly again in both of these route. Organs along the meridians will then be healed. The inflammation of the internal organs decrease. The hot regions along meridian cools or heats up to a green temperature, as observed in the thermographs that were taken.

The hot spots in the eye region, BL1, where the white colored temperature have been found to often be 2° C. higher in temperature than the normal healthier regions, which tend to be a green color (FIG. 2). Thus, if one were to interpret the thermographs on face value, one would say that the eyes are inflamed. However, none of our ASD children reports as having eye problems. With the meridian theory, it becomes clear that the hot temperature at BL1 in the corner of the eyes comes from inflammation in organs along the bladder meridian. Meridians act like optical fibers for infrared radiation and transmit the information (of inflammation of the organs along the meridians) to the observable surface point of acupoint BL1. These autistic children's parents generally report that their child has either constipation or diarrhea, some form of digestive-bowel issues. In other studies of adults we have found that hot spots in BL1 generally indicate a problem with constipation.

According to acupuncture and meridian, the hot spots above the collarbone region is ST12. Our study of adults' hot spots in the collarbone region indicates that this represents a malfunction of the thyroid. Furthermore, according to meridian theory and the practices of acupuncture, the path in which a meridian line travels affects the local area. Thus, the infrared radiation from the inflammation of thyroid gland, located above the collarbone on the left and right side of the neck, is transmitted by the stomach meridian. This would imply that autistic children might have problems with their thyroid. We, therefore, suggest that the malfunction of the thyroid be confirmed in blood tests of autistic children.

Similarly, the hot area in the armpit is where the GB22 is situated. It may also be interpreted as inflammation of the lymphatic nodes under the arm. The inflammation may be caused by the overworking of the immune system. This interpretation supports the view that an excessive function of the immune system may be a major component of ASD.

The thermographs reveal a definite heat pattern on the body surface temperature of children with autism. For practitioners that employ acupuncture or qigong to treat ASD, these thermographs provide a pictorial guidance as to where to put their needles, and where they should unblock the qi channel.

The thermographs of the abdomen of the subject in FIG. 7, images a and b show that the subject has inflammation in the colon and small intestinal areas. Furthermore, most of our autistic children presented an abnormal temperature distribution of the body surface area in the gastrointestinal and head regions. From the meridian theorists' and Chinese medicine doctors' points of view, brain disorders and gastrointestinal disorders are connected. Disorders in various parts of the body are expressed as a blockage of qi circulation along the meridian system. Removal of qi blockage along the meridian either by qigong method, acupuncture needles, herbs, or SWP will reduce the abnormality in the abdomen and brain simultaneously.

In the discussion of thermal patterns of ASD above, it is quite clear that the pattern does not depend on the meridian theory, although it does provide a rational explanation of the pattern. Hence, if such consistency of first order thermal patterns is demonstrated when more ASD children are studied in the future, then thermal patterns should be used as a diagnostic tool. Thermographs are inexpensive, non-intrusive, and non-physical.

The children may thus be used as a diagnostic tool for much younger children, say at one year, before observation of any behavioral disorder is possible. It is generally believed that the earlier the healing method is applied to children with ASD, the sooner the health problems can be helped or resolved. With early diagnosis, healing methods such as herbal supplements, qigong massage or DHW can be undertaken much earlier and with greater efficacy to stop and to prevent the development of ASD.

This was a pilot study using thermographs on thirteen children with ASD to find any abnormal thermal pattern on the body surface temperature distribution in children with ASD. We discovered a consistent set of thermal patterns that are characteristic of children with ASD. If these patterns were to remain constant for a larger sample of children with ASD, thermographs may be a promising tool to detect ASD, as well as to track the progress of improvement of ASD.

These characteristic thermal patterns, when viewed through the meridian theory, confirmed that the meridians connecting the head to the gastrointestinal system, (the gallbladder meridian, bladder meridian, stomach meridian, and small intestine meridian) are the most important meridians associated with ASD. The hot spots seen on the thermographs may provide guidance for various methods in Chinese medicine, such as where to insert needles in acupuncture treatment and where to perform qigong treatment. Specifically, we have shown that in every subject, the body's thermal pattern changed within 15 minutes after drinking SWP. There was both qualitative and quantitative evidence from thermographs and from parents' evaluation that drinking SWP improved the health of all thirteen subjects.

Finally, using quantitative analysis through health questionnaires answered by the subjects' parents we were able to conclude observe the healing effects of SWP on ASD subjects for up to three months during the trial period.

In conclusion, the method 100 for detection, treatment, and prevention of neurological development disorders utilizes an infrared imaging device, meridian theory of traditional Chinese medicine, and predetermined amounts of solid water particles to detect, treat, and prevent a neurological developmental disorder, such as autism. The method detects the disorder by initially identifying at least one hot spot on a patient with an infrared imaging device. At least one acupoint along a meridian pathway is identified. The hot spot is correlated with at least one acupoint located along the meridian pathway. The nexus of the hot spot and the meridian pathway indicates a potential neurological development disorder. A degree of irregularity of the internal organ is discerned by measuring a quantitative temperature at the nexus. A solid water particle is applied through a delivery means, such as oral, topical, vapor, and intravenous administration for treatment of the neurological developmental disorder.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What I claim is:

1. A method for detection, treatment, and prevention of a neurological development disorder, the method comprising:
determining at least one hot spot on a body with an infrared imaging device, the at least one hot spot being at least partially indicative of at least one internal organ of the body having an irregularity;
determining at least one acupoint along at least one meridian pathway, the at least one meridian pathway defined by a network that interconnects the at least one internal organ;
identifying a nexus between the at least one hot spot and the at least one acupoint;
discerning a degree of irregularity of the at least one internal organ by measuring a quantitative temperature at the nexus; and
administering a predetermined quantity of solid water particles with a delivery means, approximately to the nexus.

2. The method of claim 1, wherein the at least one meridian pathway includes at least one member selected from the group consisting of: a bladder meridian, a stomach meridian, a gallbladder meridian, a large intestine meridian, a small intestine meridian, and a tripe heater meridian.

3. The method of claim 1, wherein the at least one acupoint includes at least one member selected from the group consisting of: the left and right front center ears, the left and right inner point of the eyes, the left and right neck near the shoulder, the left and right forehead temple area, the left and right collar bone area, the left and right arm pits, and a line segment at the bottom of the neck.

4. The method of claim 1, wherein the infrared imaging device comprises a thermograph.

5. The method of claim 1, wherein the delivery means includes at least one member selected from the group consisting of: oral administration, topical administration, vapor administration, and intravenous administration.

6. The method of claim 1, wherein the oral administration comprises a liquid, a pill, or a gel consisting of the predetermined quantity of solid water particles.

7. The method of claim 6, wherein the topical administration comprises a cream consisting of the predetermined quantity of solid water particles.

8. The method of claim 7, wherein the vapor administration comprises a nebulizer and an inhaler consisting of the predetermined quantity of solid water particles.

9. The method of claim 8, wherein the intravenous administration comprises an intravenous injection consisting of the predetermined quantity of solid water particles.

10. The method of claim 9, wherein the cream is configured to be applied at least topically to at least one member selected from the group consisting of: the left and right front center ears, left and right inner points of the eyes, left and right forehead temple, left and right collar bone, and left and right arm pits.

11. The method of claim 10, wherein the step of administering a predetermined quantity of solid water particles approximately to the nexus with a delivery means, further includes administering the liquid, the pill, or the gel to a mother of a baby, who is breast fed by the mother.

12. The method of claim 11, wherein the step of administering a predetermined quantity of solid water particles approximately to the nexus with a delivery means, further includes administering the cream, the liquid, the pill, or the gel directly to the baby.

13. The method of claim 1, wherein the method further comprises at least one supplementary treatment, the at least one supplementary treatment including at least one member selected from the group consisting of: acupuncture, herbs, behavioral therapy, speech therapy, drugs, and at least one alternative method prescribed by a medical professional.

14. The method of claim 1, wherein the body comprises a body of a child.

15. The method of claim 1, wherein the neurological development disorder is an autistic spectrum disorder.

16. A method for detection, treatment, and prevention of a neurological development disorder, the method comprising:
determining at least one hot spot on a body with a thermograph, at least one hot spot being at least partially indicative of at least one internal organ of the body having an irregularity;
determining at least one acupoint along at least one meridian pathway, the at least one meridian pathway defined by a network that interconnects the at least one internal organ;
identifying a nexus between the at least one hot spot and the at least one acupoint;
discerning a degree of irregularity of the at least one internal organ by measuring a quantitative temperature at the nexus;
administering a predetermined quantity of solid water particles with a delivery means in the form of a cream, a liquid, a pill, or a gel, approximately to the nexus; and
administering at least one supplementary treatment, the at least one supplementary treatment including at least one member selected from the group consisting of:
acupuncture, herbs, behavioral therapy, speech therapy, drugs, and at least one alternative method prescribed by a medical professional.

17. The method of claim 16, wherein the delivery means includes at least one member selected from the group consisting of: oral administration, topical administration, vapor administration, and intravenous administration.

18. The method of claim 16, wherein the cream is configured to be applied at least topically to at least one member selected from the group consisting of: the left and right front center ears, left and right inner points of the eyes, left and right forehead temple, left and right collar bone, abdominal and left and right arm pits.

19. The method of claim 16, wherein the vapor administration comprises a nebulizer and an inhaler consisting of the predetermined quantity of solid water particles.

20. The method of claim 16, wherein the intravenous administration comprises an intravenous injection consisting of the predetermined quantity of solid water particle.

21. The method of claim 16, wherein the delivery means includes at least one member selected from the group consisting of: oral administration, topical administration, vapor administration, and intravenous administration.

22. The method of claim 21, wherein the vapor administration comprises a nebulizer and an inhaler consisting of the predetermined quantity of solid water particles.

23. The method of claim 21, wherein the intravenous administration comprises an intravenous injection consisting of the predetermined quantity of solid water particles.

24. A method for detection, treatment, and prevention of a neurological development disorder, the method comprising:
determining at least one acupoint along at least one meridian pathway, the at least one meridian pathway defined by a network that interconnects at least one internal organ; and
administering a predetermined quantity of solid water particles with a delivery means in the form of a cream, a liquid, a pill, or a gel, approximately to a nexus between the at least one meridian pathway and the at least one internal organ.

25. The method of claim 24, wherein the delivery means includes at least one member selected from the group consisting of: oral administration, topical administration, vapor administration, and intravenous administration.
